(19) United States
(12) Patent Application Publication
(10) Pub. No.: US 2026/0115035 A1
(43) Pub. Date: Apr. 30, 2026

(54) A FLUID COLLECTION DEVICE

(71) Applicant: Samaritan Al Private Limited, Kamataka (IN)

(72) Inventors: Anshul SINGH, New Delhi (IN); Karan RAO, Bengaluru, Karnataka (IN)

(21) Appl. No.: 19/117,010

(22) PCT Filed: Sep. 30, 2023
(86) PCT No.: PCT/IB2023/059825

§ 371 Date: Mar. 28, 2025

(30) Foreign Application Priority Data
Oct. 1, 2022 (IN) 202211048941

(51) Int. Cl. A61F 5/44; A61B 10/00; A61J 1/05

(52) U.S. Cl. CPC A61F 5/4405; A61B 10/0045; A61J 1/05

(57) ABSTRACT

The present disclosure discloses a fluid collection device configurable for improved collection, monitoring and measurement of bodily fluids for real-time analysis. The monitoring may be achieved by providing a collection vessel having a plurality of compartments that is filled with bodily fluid in a predetermined time intervals, to monitor and detect changing condition of vital organs for a longer duration.

### TECHNICAL FIELD

Present disclosure relates to a field of fluid collection systems. More specifically, the present invention relates to a fluid collection device to continuously monitor and analyse properties of the fluid collected from a user or transferred from another device in real-time.

### BACKGROUND OF THE DISCLOSURE

Bodily fluids are usually collected and measured in a timely manner to examine changing conditions of vital organs, which helps in early detection of infections, illness and the like. Conventionally, these body fluids are collected in pouches or disposable bags that are connected to a user such as a human or animal body via a catheter. The collected bodily fluids in such pouches or disposable bags is manually measured and recorded periodically by a medical practitioner or nurse. However, these conventional pouches or disposable bags are designed to collect only a limited volume of the bodily fluid and have to be replaced frequently if additional volume of bodily fluids have to be collected. Especially for a critically ailing body, it becomes important to monitor vital organs periodically to diagnose and provide appropriate medical care, hence frequent draining of the measurement chamber or disposable bags may lead to delays and monitoring of such ailing users may cause complications and loss of critical data. The fluids collected are mixed with the remaining fluid thereby diluting the concentration and mixing potentially important constituents of the fluid collected. As this method does not allow interval sampling.

Fluid sample collection with conventional methods and maintaining a log by periodically measuring the volume is prone to errors and has a high measurement error rate. The manual logs tend to also make notes for the characteristics of the fluid for example, color or cloudy nature of the fluid etc. These are difficult to detect due to the dilution of the fluid collected, for example a certain change occurring for a short period of time may go undetected as the dilution of fluid occurs.

In recent times, an automated fluid collection device which includes the pouch having an electrical system has been developed. The collected bodily fluids in such pouches are monitored and measured electronically, to provide more accurate measurements and determination of the fluid so collected. But such electronic monitoring requires additional systems to operate sensors, display units, or communications modules, which increases manufacturing and operational cost. Also, its operation is restricted to a skilled person due to complex constructions and operating systems. Further, in such devices, the pouches have a reservoir that is integrally formed within the pouches and is fluidly connected to the remaining volume of the pouch, such that, bodily fluid is initially collected in the reservoir and transferred to the remaining volume of the pouch periodically. However, additional time is consumed because of having to transfer the bodily fluid from the reservoir into the remaining volume container to begin a measurement cycle for analysis. Moreover, these automated fluid collection devices depend on external devices to perform any chemical analysis of the bodily fluids collected within the pouch which is time consuming and leading to alteration in properties of the bodily fluids, thereby causing inaccurate analysis of the fluid so collected or fouling, for example bacterial growth in collected fluid due to contact in air or during conventional sampling.

Hence, there is need for manufacturing a fluid collection device for improved analysis and early detection of ailment or infection to mitigate one or more above shortcomings.

### SUMMARY OF THE DISCLOSURE

One or more shortcomings of the prior art are overcome by a fluid collection device and a method as claimed and additional advantages are provided through the provisions of the present disclosure. Additional features and advantages are realized through the techniques of the present disclosure. Other embodiments and aspects of the disclosure are described in detail herein and are considered a part of the claimed disclosure.

In one non-limiting embodiment of the present disclosure a fluid collection device is disclosed. The fluid collection device comprises a collection vessel, at least one inlet port, a plurality of compartments and a valve assembly. The collection vessel defines a reservoir, wherein the reservoir is defined with a plurality of conduits. The at least one inlet port is defined on an inlet portion of the collection vessel, being fluidly connected to receive fluid from a user. The plurality of compartments is defined within the reservoir wherein, each compartment of the plurality of compartments is configured to receive and collect a varied amount of the fluid. The valve assembly is connectable between the at least one inlet port and the plurality of compartments. The valve assembly comprises a plurality of provisions connected to each compartment of the plurality of compartments via the plurality of conduits, wherein the valve assembly is configured to selectively allow flow of the fluid from the reservoir to the plurality of compartments at a predetermined intervals.

In an embodiment, the at least one inlet port is fluidly connected to the user by a fluid tube.

In an embodiment, the valve assembly is defined with an enclosure having one end connected to the at least one inlet port.

In an embodiment, the valve assembly comprises a timer mechanism and a flow control mechanism within the enclosure configured to selectively allow flow of the fluid from the reservoir to each of the plurality of compartments at the predetermined intervals.

In an embodiment, the timer mechanism is a discrete control mechanism.

In an embodiment, the timer mechanism is a continuous control mechanism.

In an embodiment, the flow control mechanism is defined by a cam having a flow path for the fluid from the fluid tube to one of the plurality of compartments.

In an embodiment, the timer mechanism is configured to adjust the flow control mechanism, such that each compartment of the plurality of compartments is filled with the fluid until the predetermined intervals is elapsed and a subsequent compartment is filled with the fluid.

In an embodiment, the timer mechanism comprises a biasing member having one end coupled to the flow control mechanism and another end coupled to a knob for manually loading the biasing member and to set the predetermined intervals.

In an embodiment, the fluid collection device comprises at least one control unit communicatively coupled to the fluid collection device.

In an embodiment, the at least one control unit is configured to receive one of a set of images from an image capturing device and signals from plurality of sensors disposed in the fluid collection device to measure flow rate, volume and specific gravity of the fluid.

In an embodiment, the at least one control unit is configured to compare the volume and specific gravity of the fluid with a set of predetermined values and determine a flow rate of the fluid based on inputs provided by the plurality of sensors.

In an embodiment, the at least one control unit is configured to estimate flow rate, volume, weight and specific gravity of the fluid based on comparison with the set of predetermined values.

In an embodiment, the fluid collection device comprises a hook connectable to the collection vessel at a top portion and configured to selectively expand and compress relative to the fluid collection device based on flow of the fluid into the collection vessel.

In an embodiment, the hook is a spring actuated mechanism.

In an embodiment, the plurality of sensors comprises a refractometer configured to sense refraction of the fluid and transmit at least one signal corresponding to refraction of the fluid to the at least one control unit.

In an embodiment, the at least one control unit is configured to determine specific gravity of the fluid based on the at least one signal corresponding to refraction of the fluid transmitted by the refractometer.

In an embodiment, the fluid collection device comprises a plurality of analysis strips in fluid communication with the plurality of compartments and configured to receive the fluid for analysis of the fluid.

In an embodiment, each compartment of the plurality of compartments is defined with at least one sampling port configured to allow fluid sample collection.

In another non-limiting embodiment of the present disclosure, a method for analysis of a fluid in a fluid collection device is disclosed. The method includes the steps of receiving, fluid of a user into a plurality of compartments through at least one inlet port, wherein the fluid collection device being defined with the plurality of compartments and the at least one inlet port. The at least one control unit is configured to identify volume and specific gravity of the fluid upon receiving one of a set of images captured from an image capturing device and signals from sensors disposed in the fluid collection device. The at least one control unit is configured to compare volume and the specific gravity of the fluid with a set of predetermined values. The at least one control unit is configured to determine a flow rate of the fluid, based on inputs provided by plurality of sensors. Lastly, the control unit is configured to estimate the flow rate of the fluid, and comparing with the set of predetermined values for analysing the fluid.

It is to be understood that the aspects and embodiments of the disclosure described above may be used in any combination with each other. Several of the aspects and embodiments may be combined together to form a further embodiment of the disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The novel features and characteristic of the disclosure are set forth in the appended claims. The disclosure itself, however, as well as a preferred mode ofuse, further objectives and advantages thereof, will best be understood by reference to the following detailed description of an illustrative embodiment when read in conjunction with the accompanying figures. One or more embodiments are now described, by way of example only, with reference to the accompanying figures wherein like reference numerals represent like elements and in which:

FIG. 1 illustrates a front sectional view of a fluid collection device, in accordance with some embodiments of the present disclosure;

FIG. 2 illustrates a perspective view of the device, in accordance with some embodiments of the present disclosure;

FIG. 3 illustrates a perspective view of a fluid controller and a valve assembly of the device of FIG. 1;

FIG. 4a illustrates front view of a sleeve, in accordance with some embodiments of the present disclosure;

FIG. 4b illustrates front view of a sleeve with a refractometer, in accordance with some embodiments of the present disclosure;

FIG. 5 illustrates a perspective view of a sleeve attached to the device, in accordance with some embodiments of the present disclosure;

FIG. 6a illustrates a top view of a timer mechanism, in accordance with some embodiments of the present disclosure;

FIG. 6b illustrates a front view of the timer mechanism, in accordance with some embodiments of the present disclosure;

FIG. 6c illustrates a side view of the timer mechanism, in accordance with some embodiments of the present disclosure;

FIG. 6d illustrates a perspective view of the timer mechanism, in accordance with some embodiments of the present disclosure;

FIG. 7a illustrates a perspective view of the fluid collection device, in accordance with some embodiments of the present disclosure;

FIG. 7b illustrates a top view of the device, in accordance with some embodiments of the present disclosure;

FIG. 7c illustrates a left side view of the device of FIG. 7a, in accordance with some embodiments of the present disclosure;

FIG. 7d illustrates a front view of the device of FIG. 7a, in accordance with some embodiments of the present disclosure; and

FIG. 8 illustrates a flow chart depicting a method for analysis of a fluid, in accordance with some embodiments of the present disclosure.

The figures depict embodiments of the disclosure for purposes of illustration only. One skilled in the art will readily recognize from the following description that alternative embodiments of the structure and mechanism illustrated herein may be employed without departing from the principles of the disclosure described herein.

### DETAILED DESCRIPTION

While the embodiments in the disclosure are subject to various modifications and alternative forms, specific embodiment thereof has been shown by way of example in the figures and will be described below. It should be understood, however, that it is not intended to limit the disclosure to the particular forms disclosed, but on the contrary, the disclosure is to cover all modifications, equivalents, and alternative falling within the scope of the disclosure.

The terms “comprises”, “comprising”, or any other variations thereof used in the disclosure, are intended to cover a non-exclusive inclusion, such that a device, assembly, mechanism, system, method that comprises a list of components does not include only those components but may include other components not expressly listed or inherent to such system, or assembly, or device. In other words, one or more elements in a system proceeded by “comprises . . . a” does not, without more constraints, preclude the existence of other elements or additional elements in the system or method.

Embodiments of the present disclosure discloses a fluid collection device. The fluid collection device comprises a collection vessel, at least one inlet port, a plurality of compartments and a valve assembly. The collection vessel defines a reservoir, wherein the reservoir is defined with a plurality of conduits. The at least one inlet port is defined on an inlet portion of the collection vessel, being fluidly connected to receive fluid from a user or any other external source/device. The plurality of compartments is defined within the reservoir wherein, each compartment of the plurality of compartments is configured to receive and collect a varied amount of the fluid. The valve assembly is connectable between the at least one inlet port and the plurality of compartments. The valve assembly comprises a plurality of provisions connected to each compartment of the plurality of compartments via the plurality of conduits, wherein the valve assembly is configured to selectively allow flow of the fluid from the reservoir to the plurality of compartments at a predetermined intervals. With such configuration, the fluid collection device is configurable for improved collection, monitoring and measurement of bodily fluids for real-time analysis. In some embodiments, the monitoring may be achieved by providing a collection vessel having a plurality of collection compartments that is configured to fill-up with bodily fluid at predetermined time intervals, to monitor and detect changing condition of vital organs for a longer duration. The fluid collection device allows continuous monitoring and early detection of infections or diseases, thereby aiding in precise examination using a single collection vessel.

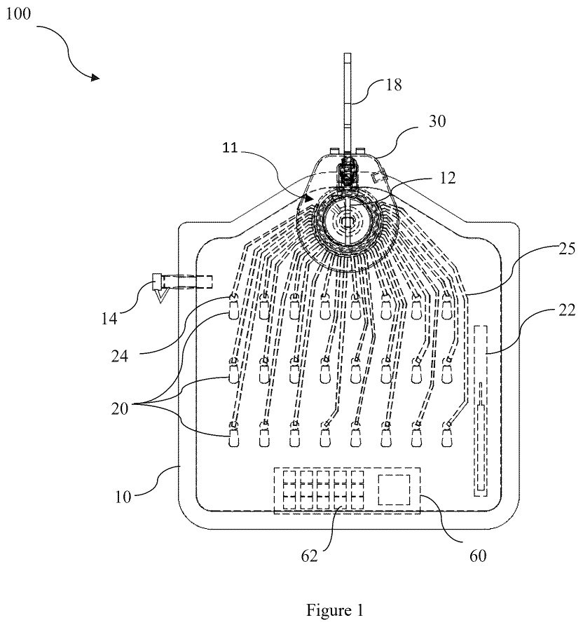

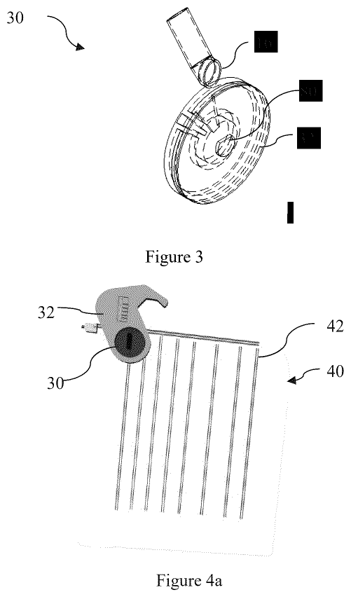

The disclosure is described in the following paragraphs with reference to FIGS. 1 to 7. In the figures, the same element or elements which have same functions are indicated by the same reference signs. One skilled in the art would appreciate that the device and the method as disclosed in the present disclosure may be used for analysis of biological fluids including but not liming to blood, urine, saliva, semen and the like. The device and the method of the present disclosure may also be implemented for analysis of other fluids including, but not limited to, water, coolants, sludge and the like without deviating from the principles of the present disclosure.

Referring now to FIGS. 1 to 3, a fluid collection device (100) [herein referred to as device] is disclosed. The device (100) may comprise a collection vessel (10) formed from a front and a rear sheet of flexible material sealed together at their edges to define a fluid reservoir. The fluid reservoir may be defined with a plurality of conduits (25) to allow flow of the fluid within the fluid reservoir. In an embodiment, the plurality of conduits (25) may be defined in a branched configuration or in a sequenced continuous configuration. In the illustrative embodiment, the plurality of conduits (25) are depicted in a branched configuration to split the fluid into multiple volumes. The collection vessel (10) defining the fluid reservoir may include at least one inlet port (12) defined on an inlet portion (11) of the collection vessel (10) for receiving fluids from a user. The collection vessel (10) may be defined with at least one outlet port (14) to facilitate drainage of fluids. Further, the at least one inlet port (12) of the collection vessel (10) may be fluidly connected to one of a fluid source and the user such as a human/animal body via a fluid tube (16). In an embodiment, the fluid tube (16) may be a catheter. Further, a valve may be provided on or along the fluid tube (16) to regulate fluid flow within the collection vessel (10). The valve is provided to prevent retrograde flow of fluid from collection vessel (10) to the fluid tube (16). In an embodiment, the collection vessel (10) may be manufactured of polymeric material or any other flexible material having requisite strength characteristic. The fluid tube (16) with its one end is connected to a catheterized user or to an external device and a second end is in fluid communication with the collection vessel (10) to direct the fluids into the collection vessel (10). In an embodiment, the fluid may be bodily fluids such as urine, blood, pus, serum, discharge, collected fluid during infection or disease etc. Other fluids could be samples from machines or the environment.

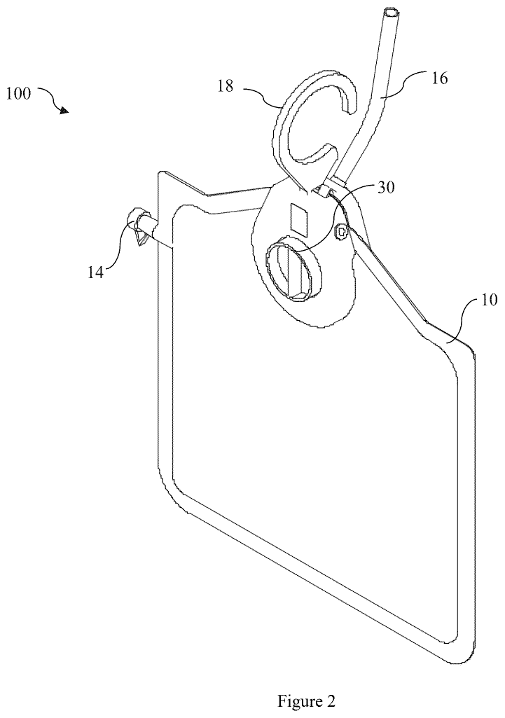

Further, the device (100) may include a plurality of compartments (20) defined with the reservoir. The plurality of compartments (20) may be arranged in an array configuration. The configuration of the plurality of conduits (25) may be based on array configuration of the plurality of compartments (20) to allow flow of the fluid from the inlet portion (11) of the collection vessel (10) to each of the plurality of compartments (20). However, such an arrangement shall not be considered as a limitation and any other requisite configuration may be used in order to serve the purpose. The plurality of compartments (20) is enclosed by the collection vessel (10) as shown in FIG. 1 to FIG. 2. Each of the plurality of compartments (20) may be configured to receive and collect a varied amount of the fluid. Further, each of the plurality of compartments (20) may be an inflatable bellow or bladder that elongates longitudinally as it gets distended upon receiving the fluid. The plurality of compartments (20) may be manufactured of latex, rubber, PVC material or any other medical grade material that serves the purpose. Each of the plurality of compartments (20) may be structured to define a predetermined volume to collect the fluid with a required flow rate. The predetermined volume of each compartment may be varied based on requirement of sample volume and design requirements of the fluid collection device (100). The collection vessel (10) may be subdivided into one or more rows, each row having some of the plurality of compartments (20).

Referring again to FIGS. 1 to 3, each of the plurality of compartments (20) may be connected to the fluid tube (16) via a valve assembly (30). The valve assembly (30) may be connectable between the at least one inlet port (12) and the plurality of compartments (20). The valve assembly (30) may be defined with an enclosure (32) having one end connected to the at least one inlet port (12). The valve assembly (30) may include a plurality of provisions, individually connected to each of the plurality of compartments (20) via the plurality of conduits (25). The valve assembly (30) may be configured to selectively allow flow of the fluid from the reservoir to the plurality of compartments (20) are a predetermined intervals. Each compartment is individually in fluid communication with the valve assembly (30). The valve assembly (30) may comprise a timer mechanism (70) and a flow control mechanism within the enclosure (32). The timer mechanism (70) and the flow control mechanism may be configured to selectively allow flow of the fluid from the reservoir to each of the plurality of compartments (20) at the predetermined intervals. The timer mechanism (70) may be employed to set a desired time interval and the flow control mechanism may allow to periodically fill each of the plurality of compartments (20) with the fluid. Here, the timer mechanism (70) may adjust the flow control mechanism, such that each compartment is filled with the fluid until a desired time interval is elapsed and a subsequent compartment is filled with the fluid, thereafter.

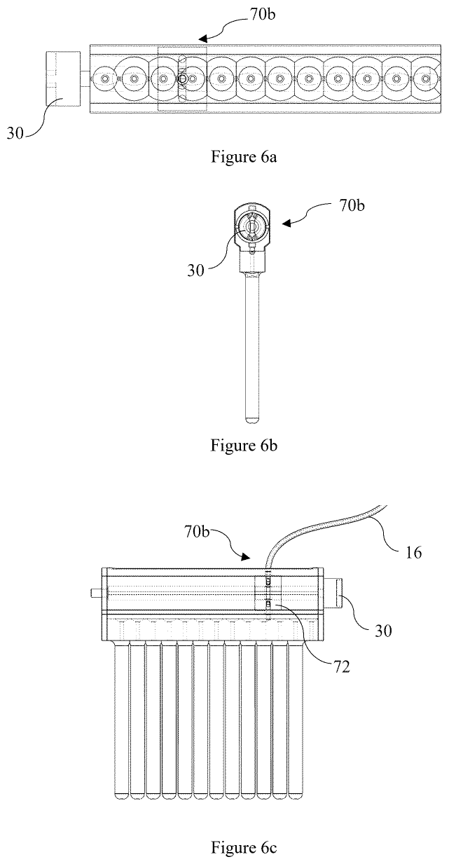

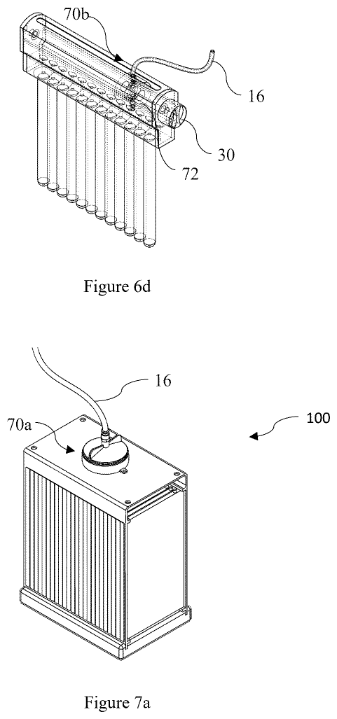

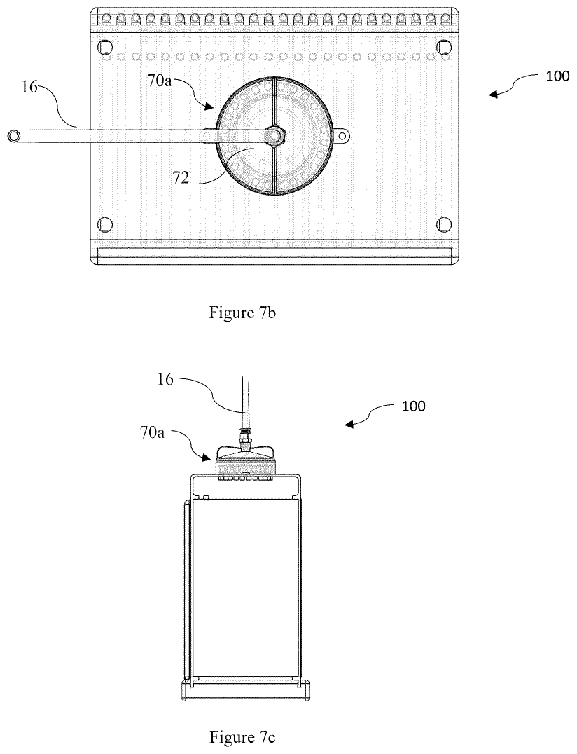

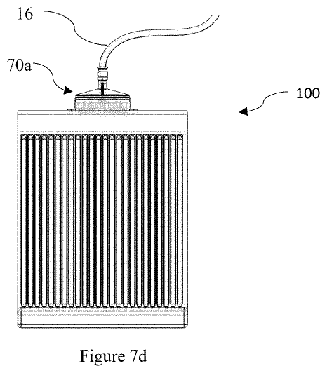

In an embodiment, the timer mechanism (70) may be either a discrete control mechanism or a continuous control mechanism. The flow control mechanism for the continuous control mechanism may be configured to align the cam (80) to direct the fluid into each of the plurality of compartments (20) for the predetermined intervals. The timer mechanism (70) may comprise a biasing member having one end coupled to the flow control mechanism and another end coupled to a knob for manually loading the biasing member and to set the desired time interval. The desired time interval may be varied by varying stiffness of the biasing member. In an embodiment, the biasing member may be a compression spring, a tension spring and the like. In an embodiment, the timer mechanism (70) may be a radial timer mechanism (70) as can be seen in FIG. 1a or a linear timer mechanism (70) as can be seen in FIGS. 6a to 6d. The radial timer mechanism (70a) may regulate the flow of the fluid into the plurality of compartments (20) as a plunger (72) of the timer mechanism (70) may rotate in a radial direction as can be seen in FIG. 7b. Whereas, the linear timer mechanism (70b) may regulate the flow of the fluid into the plurality of compartments (20) as the plunger (72) of the linear timer mechanism (70b) may traverse in linear direction along a portion of the fluid collection device (100) as can be seen in FIGS. 6a and 6c. The configuration of the plurality of conduits (25) may be varied based on the timer mechanism (70). For example, the plurality of conduits (25) may be in a radially branched configuration as can be seen in FIG. 1 corresponding to the radial timer mechanism (70a). For example, the plurality of conduits (25) may be in a linear configuration defined along the collection vessel (10) corresponding to the linear timer mechanism (70b) as can be seen in FIGS. 6c and 6d. The timer mechanism (70) may be spring loaded and can also be optionally controlled by a powered motor.

Further, the flow control mechanism may be defined by a cam (80) having a flow path for the fluid from the fluid tube (16) to one of the plurality of compartments (20), depending on a position of the cam (80). The position of the cam (80) may be adjusted while loading the biasing member of the timer mechanism (70). The desired time interval setting of the flow control mechanism may be changed, for example between 30 minutes to 60 minutes as per application requirement. In an embodiment, the flow control mechanism and the timer mechanism (70) are coupled to each other such that the fluid does not come in contact with the biasing member of the timer mechanism (70) and prevent contaminations, corrosion and damage. Thereby making the flow control mechanism as a reusable element and the collection vessel (10) as a replaceable or single use application. In an embodiment, the valve assembly (30) may comprise a weight and estimated volume scale (22) to measure a total weight/volume of fluid collected. The collection vessel (10) may be provided with a hook (18) at a top portion for it to be supported or hung, on a support structure, such as bed frame, located proximal to the user. The hook (18) may be configured to selectively expand and compress relative to the fluid collection device (100) based on flow of the fluid into the collection vessel (10). In an embodiment, extent of expansion or compression of the hook (18) relative to the fluid collection device (100) may correspond to change in volume of the fluid in the collection vessel (10). In an embodiment, the hook (18) may be a spring actuated mechanism, which may offer relative amount of spring force, which may allow the hook (18) to have flexibility.

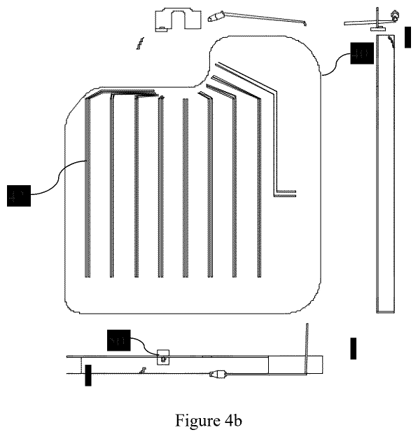

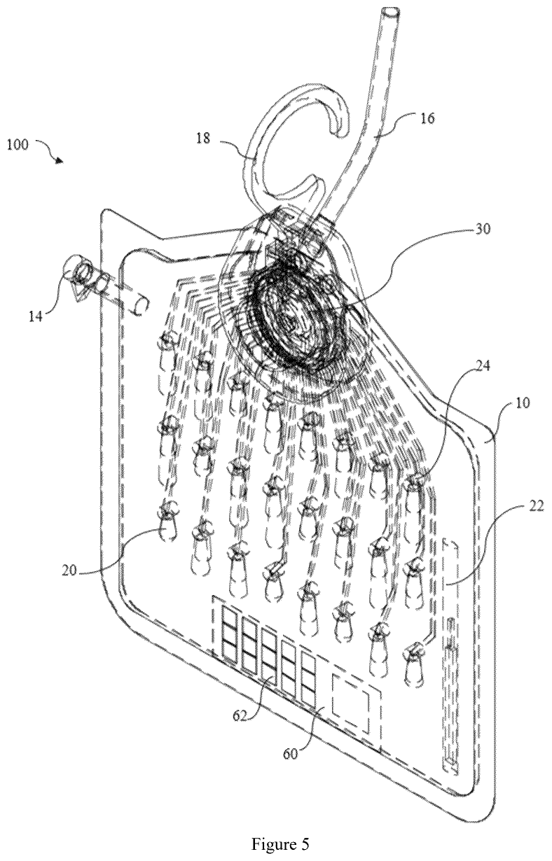

Referring now to FIGS. 4a to 5, the device (100) may comprise a sleeve (40) removably coupled or embedded to the collection vessel (10). The sleeve (40) may comprise one or more printed conductive strips (42) which may act as a capacitive transducer to electronically measure total volume/weight of fluid collected in each compartment. In an embodiment, the one or more printed conductive strips (42) may act as a non-conductive capacitive volume sensor. The printed conductive strips (42) may be communicatively coupled to at least one control unit [not shown explicitly in Figures]. The at least one control unit may be communicatively coupled to the device (100). The printed conductive strips (42) may extend along a length of the sleeve (40) so as to independently monitor fluid levels at various locations such as each compartment. Each printed conductive strip may be encapsulated in an insulating film to prevent shorting of the printed conductive strips (42) due to the fluid. Each of the printed conductive strips (42) may be electrically actuated by the at least one control unit to derive separate signals from which a differential signal is determined. This differential signal may then be compared with a predetermined maximum differential signal value to estimate a volume of fluid present in each of the compartments (20). In an embodiment, the sleeve (40) may be an add-on sleeve (40) to attach it to the collection vessel (10).

Further, the collection vessel (10) may be embedded with a volume scale (22), a digital refractometer (50) and a urinometer [not shown explicitly in Figures] to measure specific gravity and instructions to record and measure the predetermined fluid collected in the compartments (20) when it gets distended. The volume scale (22) on the collection vessel (10) may be a quantity measuring scale, ranging from 10 ml to 200 ml, with scale of 1 ml each sub scaling at 5 ml each. In an example, the urinometer scale for specific gravity measures from 1.0 to 1.04 in the urine collection vessel (10). In an embodiment, the specific gravity measured by the urinometer may be adjusted to ambient temperature for better accuracy. The gradations of volume may differ as per the intended collection fluid, ranging from a few ml to larger volumes of fluid. In an embodiment, each of the compartments (20) is provided with at least one seal port (24) to insert a needle of a syringe for fluid sample collection. These seal ports (24) enable easy and quick fluid sample collection that can be used to perform routine pathology, microscopy examination or culture sensitivity at any point of time. The at least one seal port (24) is manufactured of flexible material such as rubber and other medical grade materials and the like.

Referring now to FIGS. 7a to 7d, the device (100) may comprise a radial timer mechanism (70) coupled to the fluid tube (16) to regulate flow of the fluid into the device (100). The vessel is depicted in a cuboidal configuration as can be seen in Figure to accommodate the plurality of conduits (25) and the radial timer mechanism (70). The plurality of conduits (25) may be linearly defined along a lateral direction of the device (100) and extending vertically towards the radial timer mechanism (70) as can be seen in FIG. 7b.

In an embodiment, the at least one control unit may be communicatively coupled to the fluid collection device (100). The at least one control unit may receive one of a set of images from an image capturing device communicatively coupled to the at least one control unit. The image capturing device may be disposed in the fluid communication device (100) or may be disposed externally to the fluid collection device (100) or may be disposed in a mobile communication device such as, but not limited to, a smartphone, a computer and the like. The at least one control unit may be configured to receive the set of images from the image capturing device (100) to determine volume, flow rate, specific gravity, and weight of the fluid. In an embodiment, the device (100) may include a plurality of analysis strips (60) disposed within the collection vessel (10). Each of the analysis strips (60) may include multiple reagent coated patches (62) that serve as a chemical indicator to help detection of parameters of bodily fluids such as Leukocytes, Glucose, Ketone, Bilirubin, Blood, Specific Gravity, Protein, Urobilinogen, Nitrite, Ascorbic Acid, and pH and the like. The reagent coated patches (62), change in colour, when the fluid may come in contact with the analysis strips (60). The changed colour of the reagent coated patches (62) may be manually checked against a standard colour change reference to detect the parameters. In an embodiment, the compartments (20) may be pre-filled with reagent to instantly detect various parameters. In an embodiment, the changed colour of the reagent coated patches (62) may be captured and transmitted to the at least one control unit by the image capturing device for detecting the parameters of the fluid.

The at least one control unit may comprise, a processor and a memory unit communicatively coupled to the processor. The processors may be implemented as one or more microprocessors, microcomputers, microcontrollers, digital signal processors, central processing units, state machines, logic circuitries, and/or any devices that manipulate signals based on operational instructions. The memory unit may store processor-executable instructions, which, on execution, causes the processor to receive one or more command signals associated with the user inputs from a user interface unit coupled to the device (100). In an embodiment, the user interface unit may be coupled to the at least one control unit to receive inputs from the user for measuring the total volume of the fluid in each compartment.

Referring again to FIGS. 4a to 5, the fluid collection device (100) may comprise plurality of sensors disposed in the fluid collection device (100) to measure volume, weight and specific gravity of the fluid and transmit signals corresponding to the volume, weight and specific gravity of the fluid. The at least one control unit is configured to compare the volume and specific gravity of the fluid with a set of predetermined values. The at least one control unit may be configured to determine a flow rate of the fluid based on inputs provided by the plurality of sensors. The fluid collection device (100) may include plurality of sensors disposed in the collection vessel (10) to sense volume, weight and specific gravity of the fluid individually, where plurality of sensors measures each parameter of the volume, weight and specific gravity. The at least one control unit may be configured to estimate flow rate, volume, weight and specific gravity of the fluid based on comparison with the set of predetermined values. The plurality of sensors may include the digital refractometer (50) configured to sense refraction of the fluid and transmit at least one signal corresponding to the refraction of the fluid to the at least one control unit as can be seen in FIG. 4b. The at least one control unit may be configured to determine specific gravity of the fluid based on the at least one signal corresponding to refraction of the fluid transmitted by the refractometer (50). The real time measurement of specific gravity of the fluid, may be done via conventional digital refractometer (50) sensor systems consisting of LED diode, sensing plate and photodiode. The liquid may remain within the collection device (100) and may not come in contact with the sensor in the controller. The collection vessel (10) may comprise a window for sampling designed within the bag which couples with the digital refractometer (50) sensor on the controller. In an embodiment, the plurality of sensors may include a displacement sensor configured to measure elongation of the hook (18) and transmit at least one signal corresponding to elongation of the hook (18). The at least one control unit may be configured to determine total weight of the collection vessel (10) based on the at least one signal corresponding to the elongation of the hook (18).

In yet another embodiment of the present disclosure, the user interface unit can include a variety of software and hardware interfaces, for example, a web interface, a graphical user interface, and the like. Further, the device (100) may include a communication module that facilitates the interaction of the device (100) with an application installable on a computing device, through which an operation of the flow controller may be configured and controlled remotely. In an embodiment, the computing device includes, but is not limited to laptop computer, a desktop computer, a workstation, a mainframe computer, a server, a network server, cloud, hand-held device, wearable device and the like. The communication of the device with the computing device may occur through a wide variety of networks and protocol types, including wired networks, for example, LAN, cable, etc., and wireless networks, such as WLAN, cellular, or satellite. In an embodiment, the communication may occur via RF, Radio Frequency [RF], Bluetooth Low Energy, LoRa, ZigBee, and the like. In an embodiment, a display of the computing device may also function as the user interface unit. Further, the at least one control unit is coupled to a power source to actuate the printed capacitive strips. In an embodiment, the power source is a battery.

In an embodiment the control unit is disposed on at least one of the sleeve (40), valve assembly (30), or the collection vessel (10) or in a mobile communication device communicatively coupled to the fluid collection device (100).

In an embodiment, each of the compartments (20) comprises an outlet duct (26) connected to the plurality of analysis strips (60). The fluid in each of the compartments (20) may be selectively introduced into the analysis strips (60), by manually compressing at least one compartment (20) to force the fluids into the analysis strips (60).

In embodiment, a machine-readable optical label such as a barcode, QR code is provided on the analysis strip, which when scanned provides the standard colour change reference. Based on which, early detection of ailments may be determined.

In another embodiment, a computer vision algorithm application may be used to capture a set of images for analysing and detection of the change in colour of the reagent coated patches (62). This computer vision algorithm application corrects/normalizes a lighting condition within the captured image for accurate assessment and detection of ailments based on the detected parameters.

In an embodiment, the present device (100) is applicable for determining urine output that helps in detection of volume of urine every time interval depending upon clinical setting it is being used for. Particularly, measuring urine aids in earlier detection of infections, acute kidney injury, oliguria. Also, collecting different bodily fluids aids to understand the condition of vital organs of the user.

In another embodiment, the device (100) may facilitate collection and analysis of samples from external environmental sources such as water bodies, chemical plants and the like. Further, the device (100) may be coupled to an external apparatus that periodically and continuously supplies fluids to be tested.

In an embodiment, the design of the structure and dimensions of the device (100) may be altered based on the requirements/application.

In an embodiment, the device (100) requires minimum number of components and may be manufactured economically.

In an embodiment, the device (100) provides high accuracy and real time analysis of bodily fluids.

In an embodiment, the device (100) is compact and allows collection of bodily fluids increased volumes to monitor change in properties of bodily fluids for a longer duration.

The at least one control unit may be comprised of a processing unit. The processing unit may comprise at least one data processor for executing program components for executing user- or system-generated requests. The processing unit may be a specialized processing unit such as integrated system (bus) controllers, memory management control units, floating point units, graphics processing units, digital signal processing units, etc. The processing unit may include a microprocessor, such as AMD Athlon, Duron or Opteron, ARM's application, embedded or secure processors, IBM PowerPC, Intel's Core, Itanium, Xeon, Celeron or other line of processors, etc. The processing unit may be implemented using a mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc.

The at least one control unit may be disposed in communication with one or more memory devices (e.g., RAM, ROM etc.) via a storage interface. The storage interface may connect to memory devices including, without limitation, memory drives, removable disc drives, etc., employing connection protocols such as serial advanced technology attachment (SATA), integrated drive electronics (IDE), IEEE-1394, universal serial bus (USB), fiber channel, small computing system interface (SCSI), etc. The memory drives may further include a drum, magnetic disc drive, magneto-optical drive, optical drive, redundant array of independent discs (RAID), solid-state memory devices, solid-state drives, etc.

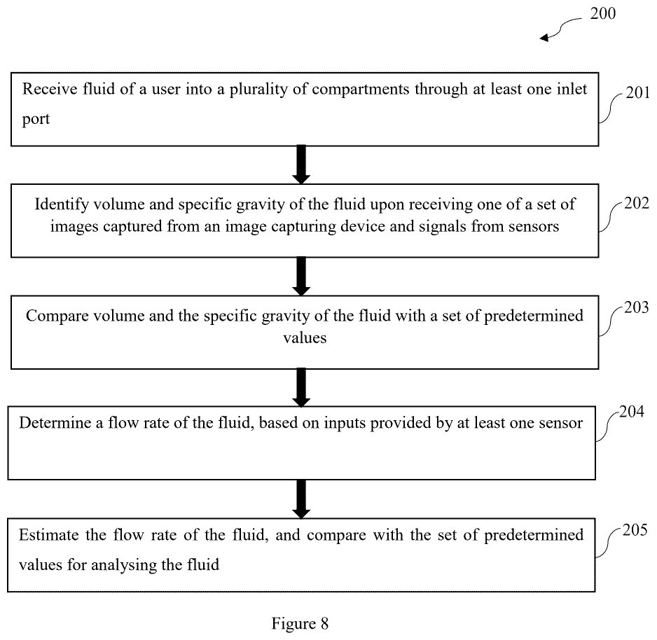

Referring now to FIG. 8 which is an exemplary embodiment of the present disclosure illustrating a method for analysis of a fluid in the fluid collection device (100).

The method may describe in the general context of processor executable instructions in the at least one control unit. Generally, the executable instructions may include routines, programs, objects, components, data structures, procedures, modules, and functions, which perform particular functions or implement particular abstract data types.

The order in which the method is described is not intended to be construed as a limitation, and any number of the described method blocks may be combined in any order to implement the method. Additionally, individual blocks may be deleted from the methods without departing from the scope of the subject matter described herein. Furthermore, the method can be implemented in any suitable hardware, software, firmware, or combination thereof.

At block 201, the fluid collection device (100) may be configured to receive fluid of the user in to the plurality of compartments (20) through the at least one inlet port (12). The at least one control unit may receive one of a set of images from an image capturing device communicatively coupled to the at least one control unit. The image capturing device may be disposed in the fluid collection device (100) or may be disposed externally to the fluid collection device (100) or may be disposed in a mobile communication device such as, but not limited to, a smartphone, a laptop and the like.

At block 202, The at least one control unit may be configured to determine volume, flow rate, specific gravity, and weight of the fluid upon receiving the set of images from the image capturing device. In an embodiment, the device (100) may include a plurality of analysis strips (60) disposed within the collection vessel (10). Each of the analysis strips (60) may include multiple reagent coated patches (62) that serve as a chemical indicator to help detection of parameters of bodily fluids such as Leukocytes, Glucose, Ketone, Bilirubin, Blood, Specific Gravity, Protein, Urobilinogen, Nitrite, Ascorbic Acid, and pH and the like. The reagent coated patches (62), change in colour, when the fluid may come in contact with the analysis strips (60). The changed colour of the reagent coated patches (62) may be manually checked against a standard colour change reference to detect the parameters. In an embodiment, the compartments (20) may be pre-filled with reagent to instantly detect various parameters. In an embodiment, the changed colour of the reagent coated patches (62) may be captured and transmitted to the at least one control unit by the image capturing device for detecting the parameters of the fluid. The fluid collection device (100) may include plurality of sensors disposed in the collection vessel (10) to sense volume, weight and specific gravity of the fluid individually, where plurality of sensors measures each parameter of the volume, weight and specific gravity.

At block 203, the at least one control unit may be configured to identify the volume, specific gravity of the fluid upon receiving signals from the plurality of sensors disposed in the fluid collection device (100). In an embodiment, the set of images from the image capturing device and the signals from the sensors may be received by the at least one control unit in real-time. At block 204, the at least one control unit may be configured to compare the volume and the specific gravity of the fluid with the set of predetermined values. The set of predetermined values may be stored in a memory associated with the at least one control unit. In an embodiment, the at least one control unit may be configured to integrate determined volume, specific gravity of the fluid with historical values of the user stored in the memory for comparison. In an embodiment, the set of predetermined values may correspond to clinical guidelines. The at least one control unit may be configured to estimate flow rate, volume, weight and specific gravity of the fluid based on comparison with the set of predetermined values at block 205. The at least one control unit may be configured to transmit alerts based on comparison of the volume and specific gravity with the set of predetermined values to indicate health risks of the user and the like.

In an embodiment, the at least one control unit may utilize machine learning models for analysis of the fluid.

### EQUIVALENTS

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### REFERENCE NUMERALS

| Description | Reference Number |
|---|---|
| Device | 100 |
| Collection vessel | 10 |
| Inlet portion | 11 |
| Inlet port | 12 |
| Outlet port | 14 |
| tube | 16 |
| Hook | 18 |
| Plurality of compartments | 20 |
| Volume scale | 22 |
| Seal port | 24 |
| Conduits | 25 |
| Outlet duct | 26 |
| valve assembly & | 30 |
| housing for refractometer, | |
| digital controller | |
| enclosure | 32 |
| Sleeve | 40 |
| Printed conductive strips | 42 |
| Refractometer | 50 |
| Plurality of analysis strips | 60 |
| Reagent coated patches | 62 |
| Timer mechanism | 70 |
| Radial timer mechanism | 70a |
| Linear timer mechanism | 70b |
| plunger | 72 |
| Cam | 80 |

We claim:

1. A fluid collection device (100), comprising:
a collection vessel (10) defining a reservoir, wherein the reservoir is defined with a plurality of conduits (25);
at least one inlet port (12) defined on an inlet portion (11) of the collection vessel (10), being fluidly connected to receive fluid from a user;
a plurality of compartments (20) defined within the reservoir wherein, each compartment (20) of the plurality of compartments (20) is configured to receive and collect a varied amount of the fluid; and
a valve assembly (30) connectable between the at least one inlet port (12) and the plurality of compartments (20), the valve assembly (30) comprising a plurality of provisions connected to each compartment of the plurality of compartments (20) via the plurality of conduits (25), wherein the valve assembly (30) is configured to selectively allow flow of the fluid from the reservoir to the plurality of compartments (20) at a predetermined intervals.

2. The fluid collection device (100) as claimed in claim 1, the at least one inlet port (12) is fluidly connected to the user by a fluid tube (16).

3. The fluid collection device (100) as claimed in claim 1, wherein the valve assembly (30) is defined with an enclosure (32) having one end connected to the at least one inlet port (12).

4. The fluid collection device (100) as claimed in claim 3, wherein the valve assembly (30) comprises a timer mechanism (70) and a flow control mechanism within the enclosure (32) configured to selectively allow flow of the fluid from the reservoir to each of the plurality of compartments (20) at the predetermined intervals.

5. The fluid collection device (100) as claimed in claim 4, the timer mechanism (70) is a discrete control mechanism.

6. The fluid collection device (100) as claimed in claim 4, the timer mechanism (70) is a continuous control mechanism.

7. The fluid collection device (100) as claimed in claim 4, wherein the flow control mechanism is defined by a cam having a flow path for the fluid from the fluid tube (16) to one of the plurality of compartments (20).

8. The fluid collection device (100) as claimed in claim 4, wherein the timer mechanism (70) is configured to adjust the flow control mechanism, such that each compartment (20) of the plurality of compartments (20) is filled with the fluid until the predetermined intervals is elapsed and a subsequent compartment (20) is filled with the fluid.

9. The fluid collection device (100) as claimed in claim 4, wherein the timer mechanism (70) comprises a biasing member having one end coupled to the flow control mechanism and another end coupled to a knob for manually loading the biasing member and to set the predetermined intervals.

10. The fluid collection device (100) as claimed in claim 1, comprises at least one control unit communicatively coupled to the fluid collection device (100).

11. The fluid collection device (100) as claimed in claim 10, wherein the at least one control unit is configured to receive one of a set of images from an image capturing device and signals from plurality of sensors disposed in the fluid collection device (100) to measure flow rate, volume and specific gravity of the fluid.

12. The fluid collection device (100) as claimed in claim 11, wherein the at least one control unit is configured to compare the volume and specific gravity of the fluid with a set of predetermined values and determine a flow rate of the fluid based on inputs provided by the plurality of sensors.

13. The fluid collection device (100) as claimed in claim 12, the at least one control unit is configured to estimate flow rate, volume, weight and specific gravity of the fluid based on comparison with the set of predetermined values.

14. The fluid collection device (100) as claimed in claim 1, comprises a hook (18) connectable to the collection vessel (10) at a top portion and configured to selectively expand and compress relative to the fluid collection device (100) based on flow of the fluid into the collection vessel (10).

15. The fluid collection device (100) as claimed in claim 10, the hook (18) is a spring actuated mechanism.

16. The fluid collection device (100) as claimed in claim 11, the plurality of sensors comprises a refractometer (50) configured to sense refraction of the fluid and transmit at least one signal corresponding to refraction of the fluid to the at least one control unit.

17. The fluid collection device (100) as claimed in claim 16, wherein the at least one control unit is configured to determine specific gravity of the fluid based on the at least one signal corresponding to refraction of the fluid transmitted by the refractometer (50).

18. The fluid collection device (100) as claimed in claim 1, comprises a plurality of analysis strips (60) in fluid communication with the plurality of compartments (20) and configured to receive the fluid for analysis of the fluid.

19. The fluid collection device (100) as claimed in claim 1, wherein each compartment of the plurality of compartments (20) is defined with at least one seal port (24) configured to allow fluid sample collection.

20. A method for analysis of a fluid in a fluid collection device (100), the method comprising:
receiving, fluid of a user into a plurality of compartments (20) through at least one inlet port (12), wherein the fluid collection device (100) being defined with the plurality of compartments (20) and the at least one inlet port (12);
identifying, by at least one control unit, volume and specific gravity of the fluid upon receiving one of a set of images captured from an image capturing device (100) and signals from sensors disposed in the fluid collection device (100);
comparing, by the at least one control unit, volume and the specific gravity of the fluid with a set of predetermined values;
determining, by the at least one control unit, a flow rate of the fluid, based on inputs provided by plurality of sensors; and
estimating, by the at least one control unit, the flow rate of the fluid, and comparing with the set of predetermined values for analysing the fluid.
